(19) United States
(12) Patent Application Publication
(10) Pub. No.: US 2013/0199723 A1
(43) Pub. Date: Aug. 8, 2013

(54) REMOVABLE TATTOO

(71) Applicant: DENISE K. GIMBEL, BOCA RATON, FL (US)

(72) Inventor: DENISE K. GIMBEL, BOCA RATON, FL (US)

(21) Appl. No.: 13/736,905

(22) Filed: Jan. 8, 2013

Related U.S. Application Data
(60) Provisional application No. 61/594,103, filed on Feb. 2, 2012.

(51) Int. Cl. A61Q 1/02; A61K 8/02

(52) U.S. Cl. CPC A61Q 1/025; A61K 8/02
USPC 156/325; 428/343; 428/34.1

(57) ABSTRACT

The removable tattoo is constructed to cover unsightly scars existing as a result of surgeries, such as tummy tucks, C-sections and the like. The removable tattoo is preferably fabricated from silicon or other durable, flexible material, and is provided with an adhesive on its rear face that permits multiple uses. The front face of the tattoo is designed to simulate a decorative, aesthetically pleasing piece of apparel, such as a stylish belt. The removable tattoo may be furnished as a kit that includes a case and a plastic tray that serves as backing for the tattoo in order to provide for storage of the tattoo when not in use.

### CROSS-REFERENCE TO RELATED APPLICATION

This application claims the benefit of U.S. Provisional Patent Application Ser. No. 61/594,103, filed Feb. 2, 2012.

### BACKGROUND OF THE INVENTION

1. Field of the Invention

The present invention generally relates to body art, and particularly to a removable tattoo utilized to cover scars and other skin blemishes.

2. Description of the Related Art

Cosmetic surgery has become a popular method to sculpt one's body to a desired configuration. Surgery to reduce the bulk of the ubiquitous “love handles” and “tummy tucks” that eliminate the unbecoming bulge of a pot belly are among the most successfully utilized cosmetic surgery techniques employed today. The downside of these cosmetic procedures is that unsightly scarring (temporary or permanent) often occurs in the abdominal area at the site of the surgical incisions. In addition, certain necessary surgeries or medical conditions, such as C-sections, wounds, the removal of moles and warts, etc., may leave embarrassing scars on the abdomen or other portions of the patient's body. While such scarring may not be visible when wearing conventional clothing, one is reluctant to don clothing (two-piece bathing suits, low-cut jeans and halters, etc.) that expose the midriff area of the body, or to expose that portion of the body in public or during intimacy. A solution to this dilemma would be to cover the scarred area with some form of body art, such as a tattoo. Many persons, however, are reluctant to undergo the needle pricks and other procedures involved to attain a tattoo. Removable art work designed to resemble a tattoo that would cover the site of the scarring would not only alleviate the dilemma of exposing unsightly scars, but would also make a fashion statement. Thus, a removable tattoo solving the aforementioned problems is desired.

### SUMMARY OF THE INVENTION

The removable tattoo is constructed to cover unsightly scars existing as a result of surgeries, such as tummy tucks, C-sections and the like. The removable tattoo is preferably fabricated from silicon or other durable material and is provided with adhesive on its rear face that permits multiple uses of the tattoo. The front face of the tattoo is designed to simulate a decorative, aesthetically pleasing piece of apparel, such as a stylish belt. The removable tattoo may be furnished in the form of a kit that includes a case that may enclose a plastic tray or rigid backing that is provided to store the tattoo when not in use.

Accordingly, the invention presents a removable tattoo that is durable, reusable, easy to clean and easy to apply. The removable tattoo is long enough to cover scars from hip to hip and will be made available in different sizes (length and width) to accommodate different waist sizes and other areas. The invention provides for improved elements thereof in an arrangement for the purposes described that are inexpensive, dependable and fully effective in accomplishing their intended purposes.

These and other features of the present invention will become readily apparent upon further review of the following specification and drawings.

### BRIEF DESCRIPTION OF THE DRAWINGS

FIG. 1 is an environmental, perspective view of a removable tattoo according to the present invention.

FIG. 2 is a perspective view of a removable tattoo according to the present invention.

FIG. 3 is a perspective view of a removable tattoo kit according to the present invention, showing the tattoo housed in a plastic case.

Similar reference characters denote corresponding features consistently throughout the attached drawings.

### DETAILED DESCRIPTION OF THE PREFERRED EMBODIMENTS

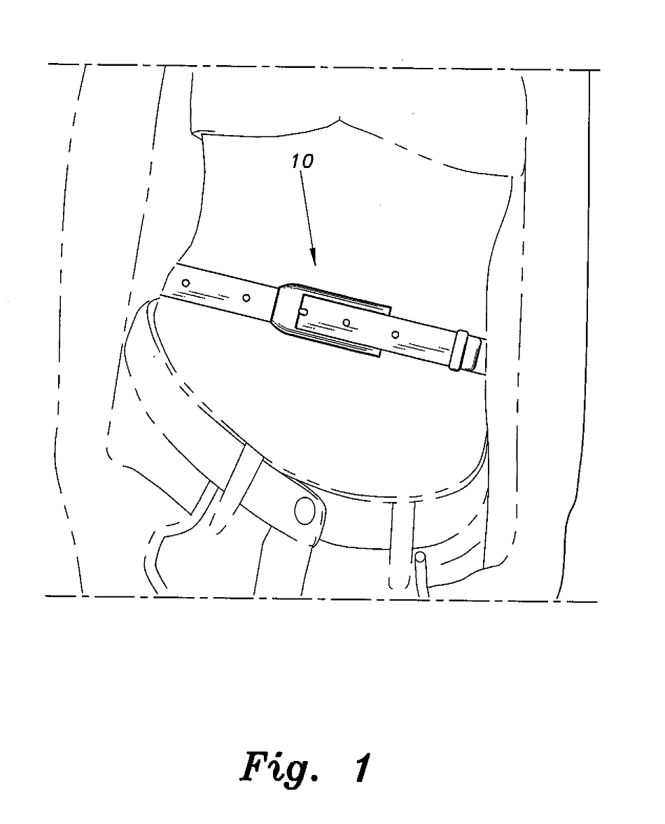

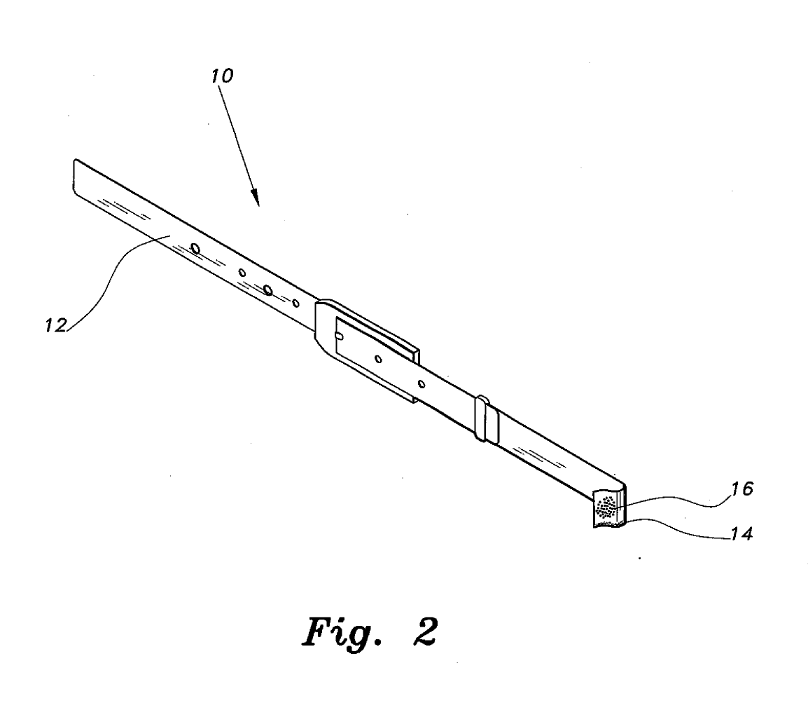

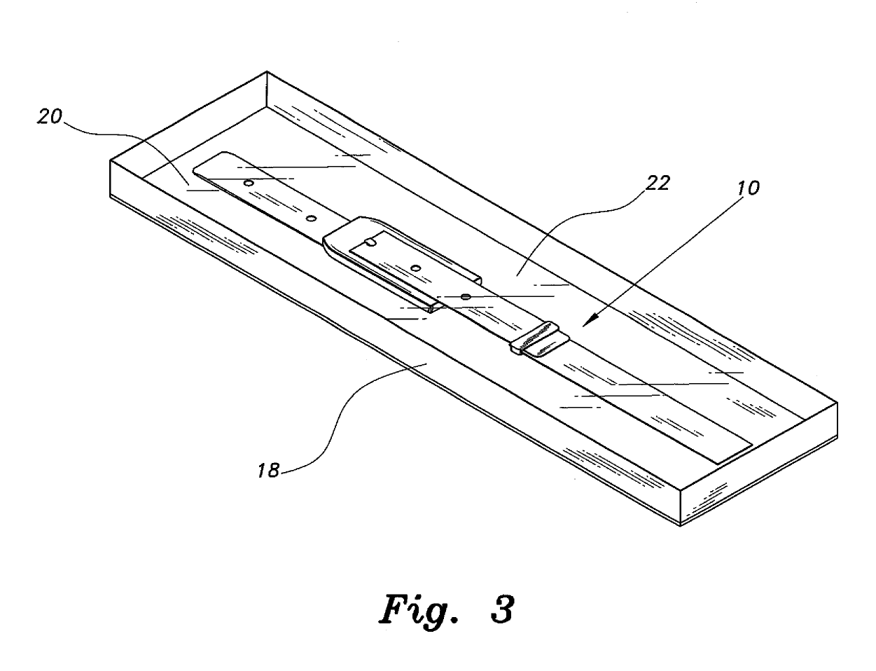

Referring to FIGS. 1 through 3, the removable tattoo 10 is preferably fabricated from silicon or other durable material so that the tattoo 10 is reusable. The material is flexible enough to allow the tattoo to conform to the curved and uneven surfaces of the wearer's body and skin and to move therewith in order to resemble a permanent tattoo, yet rugged enough to withstand repeated wear and cleansing without deterioration of the decorative design. Although the front surface 12 is illustrated herein to resemble a high-fashion belt, it should be noted that the tattoo may be designed in a myriad of styles and colors to appeal to the aesthetic tastes of various users. The rear surface 14 of the tattoo 10 is coated with a suitable, non-bioreactive, biocompatible adhesive (e.g., hydrogel) substance 16 that will remain on the rear surface through repeated applications to and removals from the wearer's body. When not in use the tattoo 10 may be housed in a plastic case 18 or the like. The case 18 may have a cover 20 to protect the tattoo 10 from accumulating dust, and may have a plastic tray or backing 22 to adhere the tattoo 10 so that the tattoo 10 does not become wrinkled or adhere to itself.

In use, the wearer simply removes the tattoo 10 from the storage tray 18 and positions the tattoo 10 across the abdominal area to cover any visible scars. Slight application of pressure by hand will cause the adhesive to securely fix the tattoo 10 in place.

It is to be understood that the present invention is not limited to the embodiments described above, but encompasses any and all embodiments within the scope of the following claims.

I claim:

1. A removable tattoo, comprising:
an elongate, decorative member having a front surface and a rear surface, the elongate member being fabricated from durable, flexible material; and
a non-bioreactive adhesive coating formed on the rear surface of the elongate member.

2. The removable tattoo according to claim 1, wherein said decorative member simulates a high-fashion belt.

3. A method of concealing a surgical scar on the abdomen, comprising the step of applying the belt tattoo of claim 2 to the abdomen to cover the surgical scar.

4. The removable tattoo according to claim 1, wherein said elongate body is made from silicon.

5. A removable tattoo kit, comprising:
an elongate flexible member having a front surface and a rear surface, the elongate member being fabricated from durable material, the front surface having a decorative front face simulating a tattoo;
a non-bioreactive pressure sensitive adhesive coating formed on the rear surface of the elongate member, whereby the flexible member is adapted for adhering to the human body;
a case defining an enclosure; and
a plastic tray disposed in the case, the elongate flexible member being attachable to the tray for storage in the case when not in use, and attachable to the human body for covering a scar when in use, the elongate flexible member being reusable for more than one application to the human body.

6. The removable tattoo kit according to claim 5, wherein said casing is provided with a removable cover.

7. The removable tattoo kit according to claim 6, wherein said durable material is silicon.

8. A method of concealing a surgical scar, comprising the step of applying a removable tattoo over the scar, the removable tattoo being a flexible sheet having a decorative front face simulating a tattoo and a rear face coated with a pressure sensitive, biocompatible adhesive.

9. The method of concealing a surgical scar according to claim 8, wherein the surgical scar is disposed on the abdomen and the decorative front face of the removable tattoo simulates a belt.

10. The method of concealing a surgical scar according to claim 8, wherein the flexible sheet is made of silicon.

11. The method of concealing a surgical scar according to claim 8, further comprising the steps of:
removing the flexible sheet from the scar;
storing the flexible sheet; and
re-applying the flexible sheet to cover the scar as needed.
